(19) United States
(12) Patent Application Publication
(10) Pub. No.: US 2008/0262452 A1
(43) Pub. Date: Oct. 23, 2008

(54) STANDALONE SMART DISPOSABLE DIAPER, KIT AND METHOD

(76) Inventors: William J. McGinnis, Cincinnati, OH (US); Scott A. Metrick, Glenview, IL (US)

Correspondence Address: LADAS & PARRY LLP, 224 SOUTH MICHIGAN AVENUE, SUITE 1600, CHICAGO, IL, 60604, US

(21) Appl. No.: 11/736,720

(22) Filed: Apr. 18, 2007

(51) Int. Cl. A61F 13/15; G08B 21/00

(52) U.S. Cl. 604/359; 340/604; 604/361; 604/389

(57) ABSTRACT

A standalone smart disposable diaper, kit and method of using same are presented for use in indicating a need to change the device when soiled. The standalone smart disposable diaper includes an inner panel, an outer panel, an absorbent pad, an absorbent composition, at least one pair of detector electrodes interposed between the inner and outer panels; a circuit coupled to the detector electrodes; a signaling beacon coupled to the circuit; and a power supply coupled to the detector electrodes, to the circuit and to the beacon. The kit includes detector electrodes, the circuit, the beacon and the power supply operatively coupled to the detector electrodes, the circuit and the beacon. The method of using the kit to convert a standard disposable diaper into a standalone smart disposable diaper device includes the steps of acquiring, allowing, inserting, observing, obtaining, removing, taping, and wearing.

### FIELD OF THE INVENTION

The present invention relates to hygienic accessories, more particularly to a standalone smart disposable diaper device, kit and method of using same for use in providing a convenient means for alerting a health care worker that a soiled diaper is in need of being changed.

### DESCRIPTION OF THE PRIOR ART

Disposable diapers have contributed significantly towards enhancing and maintaining a healthy hygienic environment for the person wearing such a device. This is primarily because the absorbent fabrics, fibers and materials now found in many commercially available disposable diapers are very effective in sequestering waste products away from the epidermis of the wearer. By sequestering the waste away from the epidermis, the conditions for inducing “diaper rash” have been significantly reduced and thus disposable diapers can enhance the hygiene of the person wearing the device.

Although disposable diapers can contribute significantly towards improving the overall health and hygiene of the wearer, it is still prudent and reasonably sound medical advise to remove a disposable diaper once it has been soiled. Because of the remarkable success in absorbing waste products by these commercially available disposable diapers, a potential problem may arise in that the health care worker may become lackadaisical with regards to ignoring a duty of diligently inspecting for soiled conditions within a timely manner of the soiling event. Paradoxically, the success of disposable diapers in being efficient absorbers of waste seems to have given rise to a new need to assure that health care workers remain diligent with regards to replacing disposable diapers when the disposable diapers are soiled.

While disposable diaper devices fulfill their respective, particular objectives and requirements, the aforementioned disposable diapers do not describe an standalone smart disposable diaper device having an inner panel, an outer panel, an absorbent pad, an absorbent composition, at least one pair of detector electrodes interposed between the inner and outer panels; a circuit coupled to the detector electrodes; a signaling beacon coupled to the circuit; and a power supply coupled to the detector electrodes, to the circuit and to the beacon. Nor do the aforementioned disposable diapers describe a kit for converting a regular commercially available disposable diaper into a standalone smart disposable diaper device, in which the kit includes detector electrodes, the circuit, the beacon and the power supply operatively coupled to the detector electrodes, the circuit and the beacon. Further none of the aforementioned disposable diaper devices disclose a method of using the kit to convert a standard disposable diaper into a standalone smart disposable diaper device includes the steps of acquiring, allowing, inserting, observing, obtaining, removing, taping, and wearing. This combination of elements would specifically match the user's particular individual needs of making it possible to provide a means for making available a disposable diaper device that can alert a health care worker that the disposable diaper device is soiled. The above-described patents make no provision for an standalone smart disposable diaper device having an inner panel, an outer panel, an absorbent pad, an absorbent composition, at least one pair of detector electrodes interposed between the inner and outer panels; a circuit coupled to the detector electrodes; a signaling beacon coupled to the circuit; and a power supply coupled to the detector electrodes, to the circuit and to the beacon. Nor do the aforementioned disposable diapers describe a kit for converting a regular commercially available disposable diaper into a standalone smart disposable diaper device, in which the kit includes detector electrodes, the circuit, the beacon and the power supply operatively coupled to the detector electrodes, the circuit and the beacon.

Therefore, a need exists for a new and improved standalone smart disposable diaper device having an inner panel, an outer panel, an absorbent pad, an absorbent composition, at least one pair of detector electrodes interposed between the inner and outer panels; a circuit coupled to the detector electrodes; a signaling beacon coupled to the circuit; and a power supply coupled to the detector electrodes, to the circuit and to the beacon. In this respect, the standalone smart disposable diaper device according to the present invention substantially departs from the conventional concepts and designs of the prior art, and in doing so provides an apparatus primarily developed for the purpose of providing a convenient means for making it possible to for alerting to a health care worker that a diaper is soiled and is in need for being changed.

### SUMMARY OF THE INVENTION

The present device, kit and method of using, according to the principles of the present invention, overcomes a number of the shortcomings of the prior art by providing a novel standalone smart disposable diaper device, kit and method for use in indicating a need to change the device when soiled. The standalone smart disposable diaper includes an inner panel, an outer panel, an absorbent pad, an absorbent composition, at least one pair of detector electrodes interposed between the inner and outer panels; a circuit coupled to the detector electrodes; a signaling beacon coupled to the circuit; and a power supply coupled to the detector electrodes, to the circuit and to the beacon. The kit includes detector electrodes, the circuit, the beacon and the power supply operatively coupled to the detector electrodes, the circuit and the beacon. The method of using the kit to convert a standard disposable diaper into a standalone smart disposable diaper device includes the steps of acquiring, allowing, inserting, observing, obtaining, removing, taping, and wearing.

In view of the foregoing disadvantages inherent in the known type standalone smart disposable diaper devices now present in the prior art, the present invention provides an improved standalone smart disposable diaper device, which will be described subsequently in great detail, is to provide a new and improved standalone smart disposable diaper device which is not anticipated, rendered obvious, suggested, or even implied by the prior art, either alone or in any combination thereof.

To attain this, the present invention essentially comprises an inner panel, an outer panel, an absorbent pad, an absorbent composition, at least one pair of detector electrodes interposed between the inner and outer panels; a circuit coupled to the detector electrodes; a signaling beacon coupled to the circuit; and a power supply coupled to the detector electrodes, to the circuit and to the beacon. The kit includes detector electrodes, the circuit, the beacon and the power supply operatively coupled to the detector electrodes, the circuit and the beacon. The method of using the kit to convert a standard disposable diaper into a standalone smart disposable diaper device includes the steps of acquiring, allowing, inserting, observing, obtaining, removing, taping, and wearing.

There has thus been outlined, rather broadly, the more important features of the invention in order that the detailed description thereof that follows may be better understood, and in order that the present contribution of the art may be better appreciated.

The invention may also include an optional thermocouple, an optional adhesive tab and an optional elastic member.

Numerous objects, features and advantages of the present invention will be readily apparent to those of ordinary skill in the art upon reading of the following detailed description of presently preferred, but nonetheless illustrative, embodiments of the present invention when taken in conjunction with the accompany drawings. In this respect, before explaining the current embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

It is therefore an aspect of the present invention to provide a new and improved standalone smart disposable diaper device that has many of the advantages of the prior standalone smart disposable diaper device and minimizing a number of their disadvantages.

It is another aspect of the present invention to provide a new and improved standalone smart disposable diaper device that may be easily and efficiently manufactured and marketed.

An even further aspect of the present invention is to provide a new and improved standalone smart disposable diaper device that has a low cost of manufacture with regard to both materials and labor, and which accordingly is then susceptible of low prices of sale to the consuming public, thereby making the standalone smart disposable diaper device economically available to the buying public.

Still another aspect of the present invention is to provide a standalone smart disposable diaper device that provides in the apparatuses and methods of the prior art some of the advantages thereof, while simultaneously overcoming some of the disadvantages normally associated therewith.

Even still another aspect of the present invention is to provide a standalone smart disposable diaper device having the interconnected elements of an inner panel, an outer panel, an absorbent pad, an absorbent composition, at least one pair of detector electrodes; a circuit; a signaling beacon; and a power supply coupled to the detector electrodes, to the circuit and to the beacon.

Still another aspect of the present invention is to provide a kit comprising the interconnected elements of the detector electrodes, the circuit, the beacon and the power supply operatively coupled to the detector electrodes, the circuit and the beacon.

Lastly, it is an object of the present invention to provide a new and improved method of using the kit to convert a standard commercially available disposable diaper into a standalone smart disposable diaper device includes the steps of acquiring, allowing, inserting, observing, obtaining, removing, taping, and wearing.

Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

These together with other objects of the invention, along with the various features of novelty that characterize the invention, are pointed out with particularity in the claims annexed to and forming a part of this disclosure. For a better understanding of the invention, its operating advantages and the specific objects attained by its uses, reference should be had to the accompanying drawings and description matter in which there are illustrated preferred embodiments of the invention.

### BRIEF DESCRIPTION OF THE DRAWINGS

The invention will be better understood and objects other than those set forth above will become apparent when consideration is given to the following detailed description thereof. Such description makes reference to the annexed drawings wherein:

FIG. 1 is a perspective view of an embodiment of the standalone smart disposable diaper device constructed in accordance with the principles of the present invention;

FIG. 2 is a cross sectional view of a standalone smart disposable diaper device of the present invention;

FIG. 3 is a perspective view of an embodiment of the standalone smart disposable diaper device of the present invention;

FIG. 4A-D are top and side views of four respective embodiments of a kit for a standalone smart disposable diaper device of the present invention; and

FIG. 5 is a perspective view of a kit for a standalone smart disposable diaper device mounted onto a conventional disposable diaper.

The same reference numerals refer to the same parts throughout the various figures.

### DETAILED DESCRIPTION OF THE PREFERRED EMBODIMENT

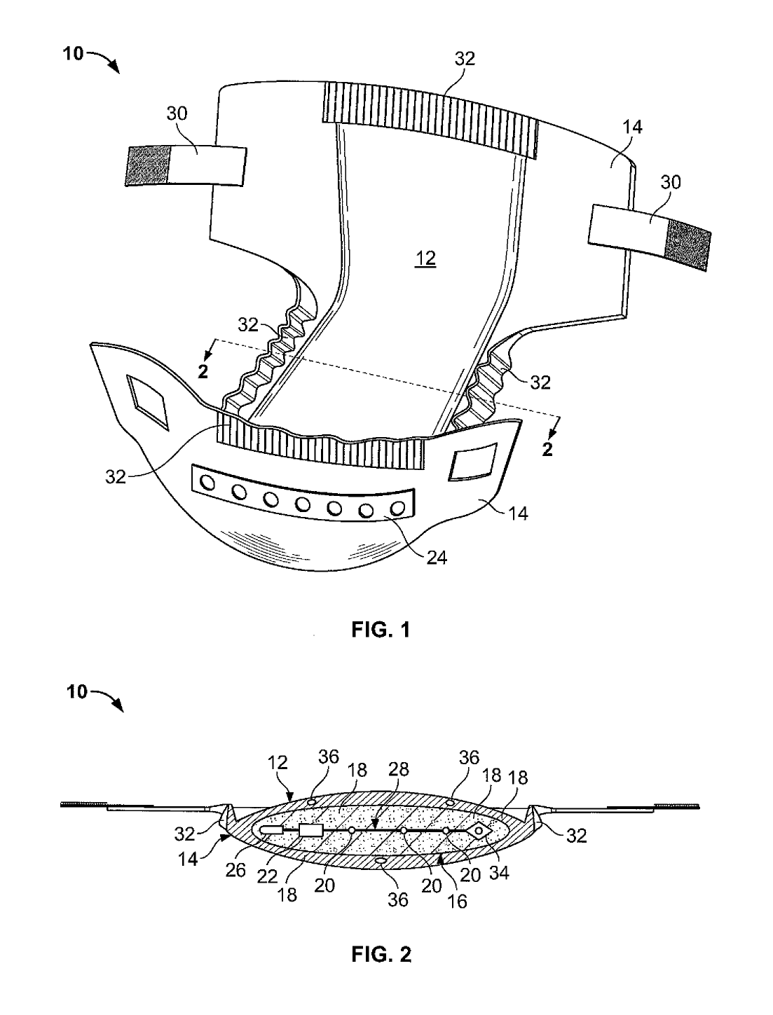

Referring now to the drawings, and in particular FIGS. 1 to 5 thereof, one preferred embodiment of the present invention is shown and generally designated by the reference numeral 10. One preferred embodiment of a standalone smart disposable diaper device 10 for use in indicating a need to change the standalone smart disposable diaper device 10 comprises: an inner panel 12, an outer panel 14, an absorbent pad 16, an absorbent composition 18, at least one pair of detector electrodes 20, a circuit 22, a beacon 24, and a power supply 26. The inner panel 12 being configured to be liquid pervious. The outer panel 14 is attached to the inner panel 12 in which the outer panel 14 being configured to be substantially liquid impervious. The absorbent pad 16 is configured to be interposed between the inner and outer panel (12 and 14, respectively) in which the absorbent pad 16 is in fluid communications with the inner panel 12. The absorbent composition 18 is distributed onto and throughout the pad 16. The detector electrodes 20 are interposed between the inner and outer panel (12 and 14, respectively). The circuit 22 is operatively coupled to the detector electrodes 20. The beacon 24 is operatively coupled to the circuit 22. The power supply 26 is coupled to the detector electrodes 20, the circuit 22 and the beacon 24.

The outer panel 14 may be any commercially available outer panel 14 as long as the outer panel 14 is substantially liquid impervious. Accordingly, the outer panel 14 may be selected from the group consisting of polypropylene, polyamide, polyester, polyethylene, ethylene-vinyl acetate, polyurethane, polyolefin blends, co-polyesters, block copolymers and admixtures thereof.

The absorbent pad 16 may be any commercially available absorbent pad 16 such as those being fabricated of materials selected from members of the group consisting of fluffed cellulose fibers, textile fibers, web of polymeric fibers, wood pulp fibers, polyester, polypropylene, polyurethane, cellulose sponge, and hydrophilic synthetic sponge.

The absorbent composition 18 may be any commercially available absorbent composition such as those made from a superabsorbent polymer selected from the group consisting of polyacrylate polymers, starch graft copolymers, cellulose graft copolymers, cross-linked carboxymethylcellulose derivatives, and admixtures thereof.

The detector electrodes 20 may be mounted anywhere within the device 10 as long as they are interposed between the inner and outer panel (12 and 14, respectively). One preferred embodiment is that the detector electrodes 20 are attached to the adsorbent pad 16. Another preferred embodiment is that the detector electrodes 20 are attached to the outer panel 14.

The circuit 22 may be mounted anywhere within the device 10 as long as they are interposed between the inner and outer panel (12 and 14, respectively). One preferred embodiment is that the circuit 22 is attached to the outer panel 14. Another preferred embodiment is that the circuit 22 is attached to the outer panel 14.

The beacon 24 may be mounted anywhere within the device 10 as long as they are interposed between the inner and outer panel (12 and 14, respectively). One preferred embodiment is that the beacon 24 is attached to the outer panel 14. Another preferred embodiment is that the beacon 24 is attached to the outer panel 14. The beacon 24 may be any commercially available beacon 24 such as an electromagnetic speaker beacon 24, a piezoelectric speaker beacon 24, a light emitting diode beacon 24, a liquid crystal diode beacon 24, and an incandescent lamp beacon 24.

The power supply 26 may be mounted anywhere within the device 10 as long as they are interposed between the inner and outer panel (12 and 14, respectively). One preferred embodiment is that the power supply 26 is attached to the outer panel 14. Another preferred embodiment is that the power supply 26 is attached to the outer panel 14. The power supply 26 may be any commercially available power supply 26 such as a battery and a high capacity capacitor. One preferred embodiment is that the power supply 26 is a battery selected from the group consisting of a zinc-carbon battery, zinc-chloride battery, an alkaline/manganese battery, a silver-oxide battery, a lithium battery, a mercury battery, and a water-activated battery. Another preferred embodiment of the power supply 26 is composed of a water-activated battery having an anode selected from a magnesium anode, and a magnesium-zinc alloy, the water-activated battery having a cathode selected from the group consisting of silver chloride, cuprous chloride, cuprous bromide, cuprous iodide, and cuprous thiocyanate, copper sulfate, and manganese dioxide.

An optional networking 28 of interconnected detector electrodes 20 may be added to the device 10 in which each detector electrode 20 of the networking 28 is operatively coupled to the circuit 22.

An optional adhesive tab 30 may be added to the device 10 in which the optional adhesive tab 30 is attached to the outer panel 14.

An optional elastic member 32 may be added to the device 10 in which the optional elastic member 32 is bonded under tension to the outer panel 14.

An optional thermocouple 34 may be added to the device 10 wherein the optional thermocouple 34 is interposed between the inner and outer panel (12 and 14, respectively) in which the optional thermocouple 34 is operatively coupled to the circuit 22.

An optional fragrant agent 36 may be added to the device 10 in which the fragrant agent 36 may be selected from the group consisting of apple essence, balsam essence, benzoin resin, blueberry essence, cassia oil, cedar oil, cinnamon essence, clove oil, coriander essence, eucalyptus essence, fresh peach essence, jasmine essence, labdanum resin, lavender essence, lemon essence, lemon oil, musk essence, nutmeg essence, olibanum resinoid, orange oil, patchouli essence, peppermint essence, Peru balsam, pine oil, raspberry essence, rose extract, sandalwood oil, spearmint essence, styrax, vanilla essence, wintergreen essence, 4-acetyl-6-tert-butyl-1,1-dimethylindane, 5-acetyl-3-isopropyl-1,1,2,6-tetramethylindane, 6-acetyl-1,1,2,3,3,5-hexamethylindane, 7-acetyl-1,1,3,4,4,6-hexamethyltetralin, 7-acetyl-1,2,3,4,5,6,7,8-octahydro-1,1,6,7-tetramethylnaphthalene, ambroxane, amylcinnamaldehyde, anisaldehyde, benzophenone, benzyl acetate, benzyl salicylate, caryophyllene alcohol, cedrol, cedryl acetate, condensation products of hydroxycitronellal and methyl anthranilate, condensation products of hydroxycitronellal and indole, condensation products of phenylacetaldehyde and indole, coumarin, cyclopentadecanolide, γ-decalactone, 2-(1,1-dimethylethyl)cyclo-hexanol acetate, dodecahydro-3a,6,6,9a-tetramethylnaphtho[2,1b]furan, 1-dodecanal, 2-ethyl-4-(2,2,3-trimethyl-3-cyclopenten-1-yl)-2-buten-1-ol, ethylvanillin, formyltricyclodecane, geraniol, heliotropin, hexylcinnamaldehyde, 4-(4-hydroxy-4-methylpentyl)-3-cyclohexene-1-carboxaldehyde, 7-hydroxy-3,7-dimethyloctanal, 16-hydroxy-9-hexadecenoic acid lactone, 1,3,4,6,7,8-hexahydro-4,6,6,7,8,8-hexamethylcyclopenta-γ-2-benzopyran, hydroxyphenylbutanone, α-ionone, β-ionone, γ-ionone, isohexenylcyclohexylcarboxaldehyde, α-isomethylionone, linalyl acetate, 2-methyl-2-(isopropylphenyl)propionaldehyde, 2-methyl-2-(para-isopropylphenyl)propionaldehyde, 2-methyl-3-para-tert-butylphenyl)propionaldehyde, 2-methyl-3-(tert-butylphenyl)propionaldehyde, methyl 1,6,10-trimethyl-2,5,9-cyclododecatrien-1-yl ketone, methylcedrylone, methyl dihydrojasmonate, methyl β-naphthyl ketone, methyl γ-naphthyl ketone, β-naphthol methyl ether, nerol, para-tert-butylcyclohexyl acetate, tert-butylcyclohexyl acetate, tricyclodecenyl propionate, tricyclodecenyl acetate, 5-(2,2,3-trimethylcyclopent-3-enyl)-3-methylpentan-2-ol, tricyclodecenyl acetate, tricyclodecenyl propionates, phenylethyl alcohol, terpineol, linalool, and 10-undecen-1-al.

One preferred embodiment of a standalone smart disposable diaper kit 38 for use in converting a disposable diaper 40 into a standalone smart diaper device 10 comprises: at least one pair of detector electrodes 20, a circuit 22 a beacon 24, and a power supply 26. The circuit 22 is configured to be operatively coupled to the detector electrodes 20. The beacon 24 is configured to be operatively coupled to the circuit 22. Finally, the power supply 26 is configured to be operatively coupled to the detector electrodes 20, the circuit 22 and the beacon 24. The kit 38 is configured to have a distal and proximate ends (42 and 44, respectively) in which the distal end 42 of the kit 38 houses the detector electrodes 20 and the proximate end 44 houses the beacon 24.

An optional thermocouple 34 may be added to the kit 38 in which the optional thermocouple 34 is configured to be operatively coupled to the circuit 22.

One preferred embodiment of a method of using a kit 38 to convert a disposable diaper 40 into a standalone smart disposable diaper device 10 comprises the steps of acquiring, allowing, inserting, observing, obtaining, removing, taping, and wearing. The obtaining step comprises obtaining the kit 38 comprising: at least one pair of detector electrodes 20; a circuit 22 operatively coupled to the detector electrodes 20; a beacon 24 operatively coupled to the circuit 22; and a power supply 26 operatively coupled to the detector electrodes 20, the circuit 22 and the beacon 24 wherein the kit 38 having a distal and proximate ends (42 and 44, respectively) in which the distal end 42 of the kit 38 housing the detector electrodes 20 and the proximate end 44 housing the beacon 24. The acquiring step comprises acquiring an unsoiled disposable diaper 40. The inserting step comprises inserting the distal end 42 of the kit 38 into the acquired disposable diaper 40. The taping step comprises taping the proximate end 44 of the kit 38 onto an outer panel 14 of the disposable diaper 40 wherein the steps of inserting and taping result in converting the disposable diaper 40 into a standalone smart disposable diaper device 10. The wearing step comprises wearing the standalone smart disposable diaper device 10. The soiling step comprises soiling the standalone smart disposable diaper device 10. The allowing step comprises allowing the circuit 22 to sense an increase in conductivity between the detector electrodes 20 when the standalone smart disposable diaper device 10 is soiled. The observing step comprises observing an alarm signal emanating from the beacon 24 when the circuit 22 senses the increase in conductivity and activates the alarm signal. The removing step comprises removing the soiled standalone smart disposable diaper device 10.

Referring now to FIG. 1 which depicts a perspective view of an embodiment of the standalone smart disposable diaper device 10 showing an inner panel 12; an outer panel 14 attached to the inner panel 12; a beacon 24 attached to the outer panel 14; a plurality of adhesive tabs 30 attached to the outer panel 14; and a plurality of elastic members 32 bonded under tension to the outer panel 14.

Referring now to FIG. 2 which depicts a cross sectional view of a standalone smart disposable diaper device 10 showing an outer panel 14 attached to the inner panel 12; an absorbent pad 16 interposed between the inner and outer panels (12 and 14, respectively); an absorbent composition 18 distributed onto the pad 16; detector electrodes 20 interposed between the inner and outer panels (12 and 14, respectively) and mounted within the adsorbent pad 16; a circuit 22 operatively coupled to the detector electrodes 20; a power supply 26 shown operatively coupled to the detector electrodes 20, and the circuit 22; a fragrant agent 36 interposed between the inner and outer panels (12 and 14, respectively); and a plurality of elastic members 32 bonded under tension to the outer panel 14.

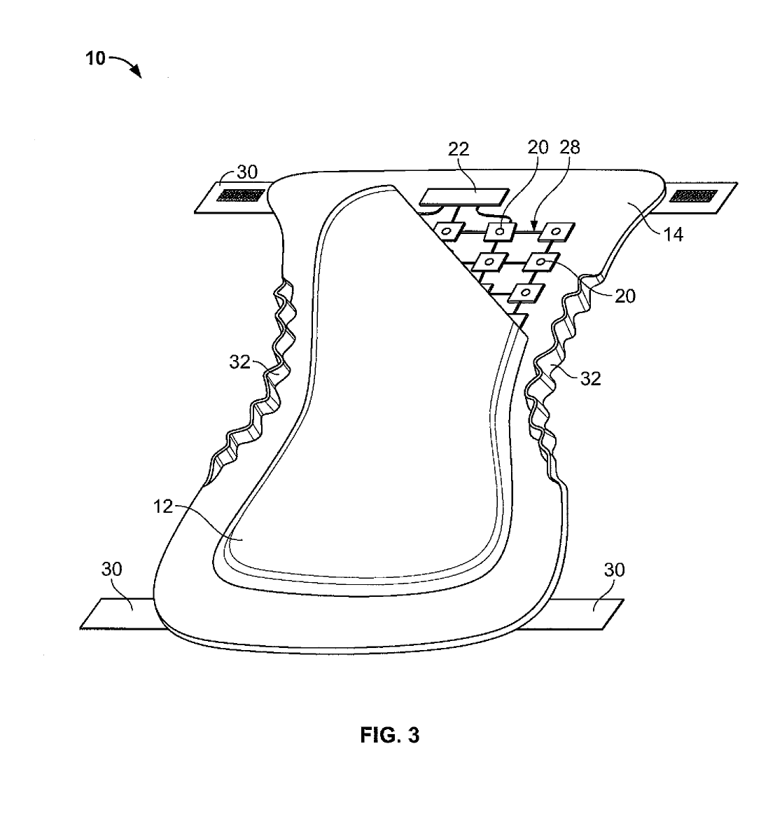

Referring now to FIG. 3 which depicts a perspective view of an embodiment of the standalone smart disposable diaper device 10 illustrating an outer panel 14 attached to the inner panel 12; a networking 28 of interconnected detector electrodes 20 in which each detector electrode 20 is shown operatively coupled to the circuit 22; a plurality of adhesive tabs 30 attached to the outer panel 14; and a plurality of elastic members 32 bonded under tension to the outer panel 14.

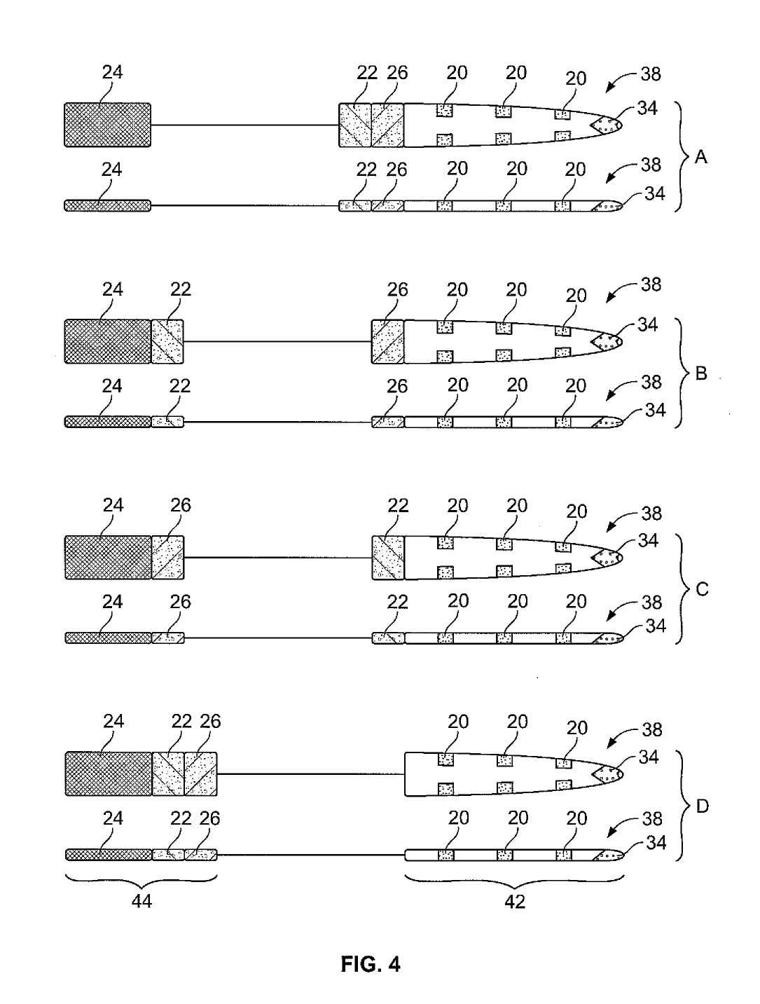

Referring now to FIGS. 4A-D which depict top and side views of four respective embodiments of a kit 38 showing the power supply 26 operatively coupled to the detector electrodes 20, to the circuit 22 and to the beacon 24, in which the kit 38 is shown having a distal and proximate ends (42 and 44, respectively) in which the distal end 42 of the kit 38 houses the detector electrodes 20 and the proximate end 44 housing the beacon 24.

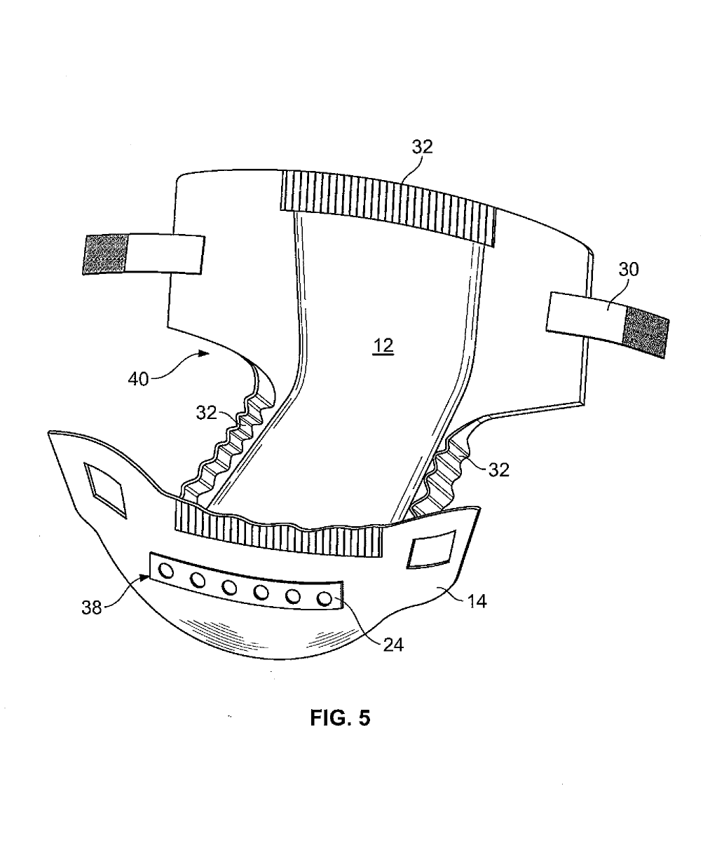

Referring now to FIG. 5 is a perspective view of a kit 38 of the present invention mounted within a conventional disposable diaper 40. The beacon 24 of the kit 38 is shown with the remaining components sequestered within the conventional disposable diaper 40. The conventional disposable diaper 40 is shown having an outer panel 14 attached to the inner panel 12; a plurality of adhesive tab 30 attached to the outer panel 14; and a plurality of elastic members 32 bonded under tension to the outer panel 14.

As to the manner of usage and operation of the present invention, the same should be apparent from the above description. Accordingly, no further discussion relating to the manner of usage and operation will be provided.

While a preferred embodiment of the standalone smart disposable diaper device has been described in detail, it should be apparent that modifications and variations thereto are possible, all of which fail within the true spirit and scope of the invention. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Throughout this specification, unless the context requires otherwise, the word “comprise” or variations such as “comprises” or “comprising” or the term “includes” or variations, thereof, or the term “having” or variations, thereof will be understood to imply the inclusion of a stated element or integer or group of elements or integers but not the exclusion of any other element or integer or group of elements or integers. In this regard, in construing the claim scope, an embodiment where one or more features is added to any of the claims is to be regarded as within the scope of the invention given that the essential features of the invention as claimed are included in such an embodiment.

Those skilled in the art will appreciate that the invention described herein is susceptible to variations and modifications other than those specifically described. It is to be understood that the invention includes all such variations and modification which fall within its spirit and scope. The invention also includes all of the steps, features, compositions and compounds referred to or indicated in this specification, individually or collectively, and any and all combinations of any two or more of said steps or features.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

We claim:

1. A standalone smart disposable diaper device for use in indicating a need to change the standalone smart disposable diaper device, said device comprising:
an inner panel being liquid pervious;
an outer panel attached to the inner panel, the outer panel being substantially liquid impervious;
an absorbent pad interposed between the inner and outer panels, the absorbent pad in fluid communications with the inner panel;
an absorbent composition distributed onto the pad;
at least one pair of detector electrodes interposed between the inner and outer panels;
a circuit operatively coupled to the detector electrodes;
a beacon operatively coupled to the circuit; and
a power supply operatively coupled to the detector electrodes, the circuit and the beacon.

2. The device of claim 1 further comprising a networking of interconnected detector electrodes wherein each detector electrode being operatively coupled to the circuit.

3. The device of claim 1 further comprising at least one adhesive tab attached to the outer panel.

4. The device of claim 1 further comprising at least one elastic member bonded under tension to the outer panel.

5. The device of claim 1 further comprising a thermocouple interposed between the inner and outer panels, the thermocouple operatively coupled to the circuit.

6. The device of claim 1 further comprising a fragrant agent interposed between the inner and outer panels, the fragrant agent selected from the group consisting of apple essence, balsam essence, benzoin resin, blueberry essence, cassia oil, cedar oil, cinnamon essence, clove oil, coriander essence, eucalyptus essence, fresh peach essence, jasmine essence, labdanum resin, lavender essence, lemon essence, lemon oil, musk essence, nutmeg essence, olibanum resinoid, orange oil, patchouli essence, peppermint essence, Peru balsam, pine oil, raspberry essence, rose extract, sandalwood oil, spearmint essence, styrax, vanilla essence, wintergreen essence, 4-acetyl-6-tert-butyl-1,1-dimethylindane, 5-acetyl-3-isopropyl-1,1,2,6-tetramethylindane, 6-acetyl-1,1,2,3,3,5-hexamethylindane, 7-acetyl-1,1,3,4,4,6-hexamethyltetralin, 7-acetyl-1,2,3,4,5,6,7,8-octahydro-1,1,6,7-tetramethylnaphthalene, ambroxane, amylcinnamaldehyde, anisaldehyde, benzophenone, benzyl acetate, benzyl salicylate, caryophyllene alcohol, cedrol, cedryl acetate, condensation products of hydroxycitronellal and methyl anthranilate, condensation products of hydroxycitronellal and indole, condensation products of phenylacetaldehyde and indole, coumarin, cyclopentadecanolide, γ-decalactone, 2-(1,1-dimethylethyl)cyclo-hexanol acetate, dodecahydro-3a,6,6,9a-tetramethylnaphtho[2,1b]furan, 1-dodecanal, 2-ethyl-4-(2,2,3-trimethyl-3-cyclopenten-1-yl)-2-buten-1-ol, ethylvanillin, formyltricyclodecane, geraniol, heliotropin, hexylcinnamaldehyde, 4-(4-hydroxy-4-methylpentyl)-3-cyclohexene-1-carboxaldehyde, 7-hydroxy-3,7-dimethyloctanal, 16-hydroxy-9-hexadecenoic acid lactone, 1,3,4,6,7,8-hexahydro-4,6,6,7,8,8-hexamethylcyclopenta-γ-2-benzopyran, hydroxyphenylbutanone, α-ionone, β-ionone, γ-ionone, isohexenylcyclohexylcarboxaldehyde, α-isomethylionone, linalyl acetate, 2-methyl-2-(isopropylphenyl)propionaldehyde, 2-methyl-2-(para-isopropylphenyl)propionaldehyde, 2-methyl-3-(para-tert-butylphenyl)propionaldehyde, 2-methyl-3-(tert-butylphenyl)propionaldehyde, methyl 1,6,10-trimethyl-2,5,9-cyclododecatrien-1-yl ketone, methylcedrylone, methyl dihydrojasmonate, methyl β-naphthyl ketone, methyl γ-naphthyl ketone, β-naphthol methyl ether, nerol, para-tert-butylcyclohexyl acetate, tert-butylcyclohexyl acetate, tricyclodecenyl propionate, tricyclodecenyl acetate, 5-(2,2,3-trimethylcyclopent-3-enyl)-3-methylpentan-2-ol, tricyclodecenyl acetate, tricyclodecenyl propionates, phenylethyl alcohol, terpineol, linalool, and 10-undecen-1-al.

7. The device of claim 1 wherein the detector electrodes are is attached to the outer panel.

8. The device of claim 1 wherein the detector electrodes are attached to the adsorbent pad.

9. The device of claim 1 wherein the circuit is attached to the outer panel.

10. The device of claim 1 wherein the circuit is attached to the adsorbent pad.

11. The device of claim 1 wherein the power supply is attached to the outer panel.

12. The device of claim 1 wherein the power supply is attached to the adsorbent pad.

13. The device of claim 1 wherein the beacon is attached to the adsorbent pad.

14. The device of claim 1 wherein the beacon is attached to the outer panel.

15. The device of claim 1 wherein the detector electrodes are attached to the adsorbent pad.

16. The device of claim 1 wherein the detector electrodes are attached to the outer panel.

17. The device of claim 1 wherein the beacon is selected from the group consisting of an electromagnetic speaker beacon, a piezoelectric speaker beacon, a light emitting diode beacon, a liquid crystal diode beacon, and an incandescent lamp beacon.

18. The device of claim 1 wherein the power supply is selected from the group consisting of a battery and a high capacity capacitor.

19. The device of claim 18 wherein the battery is selected from the group consisting of a zinc-carbon battery, zinc-chloride battery, an alkaline/manganese battery, a silver-oxide battery, a lithium battery, a mercury battery, and a water-activated battery.

20. The device of claim 19 wherein the water-activated battery having an anode selected from a magnesium anode, and a magnesium-zinc alloy, the water-activated battery having a cathode selected from the group consisting of silver chloride, cuprous chloride, cuprous bromide, cuprous iodide, and cuprous thiocyanate, copper sulfate, and manganese dioxide.

21. The device of claim 1 wherein the absorbent pad being fabricated of materials selected from members of the group consisting of fluffed cellulose fibers, textile fibers, web of polymeric fibers, wood pulp fibers, polyester, polypropylene, polyurethane, cellulose sponge, and hydrophilic synthetic sponge.

22. The device of claim 1 wherein the outer panel being selected from the group consisting of polypropylene, polyamide, polyester, polyethylene, ethylene-vinyl acetate, polyurethane, polyolefin blends, co-polyesters, block copolymers and admixtures thereof.

23. The device of claim 1 wherein the absorbent composition being a superabsorbent polymer selected from the group consisting of polyacrylate polymers, starch graft copolymers, cellulose graft copolymers, cross-linked carboxymethylcellulose derivatives, and admixtures thereof.

24. A standalone smart disposable diaper kit for use in converting a disposable diaper into a standalone smart diaper device, said kit comprising:
at least one pair of detector electrodes;
a circuit operatively coupled to the detector electrodes;
a beacon operatively coupled to the circuit; and
a power supply operatively coupled to the detector electrodes, the circuit and the beacon, wherein the kit having a distal and proximate ends in which the distal end of the kit housing the detector electrodes and the proximate end housing the beacon.

25. The kit of claim 24 further comprising a thermocouple operatively coupled to the circuit.

26. A method of using a kit to convert a disposable diaper into a standalone smart disposable diaper device, the method comprising the steps of:
obtaining the kit comprising:
at least one pair of detector electrodes;
a circuit operatively coupled to the detector electrodes;
a beacon operatively coupled to the circuit; and
a power supply operatively coupled to the detector electrodes, the circuit and the beacon wherein the kit having a distal and proximate ends in which the distal end of the kit housing the detector electrodes and the proximate end housing the beacon;
acquiring an unsoiled disposable diaper;
inserting the distal end of the kit into the acquired disposable diaper;
taping the proximate end of the kit onto an outer panel of the disposable diaper wherein the steps of inserting and taping result in converting the disposable diaper into a standalone smart disposable diaper device;
wearing the standalone smart disposable diaper device;
soiling the standalone smart disposable diaper device;
allowing the circuit to sense an increase in conductivity between the detector electrodes when the standalone smart disposable diaper device is soiled;
observing an alarm signal emanating from the beacon when the circuit senses the increase in conductivity and activates the alarm signal; and
removing the soiled standalone smart disposable diaper device.
